(19) United States
(12) Patent Application Publication
(10) Pub. No.: US 2023/0144367 A1
(43) Pub. Date: May 11, 2023

(54) MECHANICAL-BOND-PROTECTED STABLE BISRADICALS

(71) Applicant: Northwestern University, Evanston, IL (US)

(72) Inventors: Kang Cai, Evanston, IL (US); James Fraser Stoddart, Evanston, IL (US)

(21) Appl. No.: 17/907,374

(22) PCT Filed: Mar. 25, 2021
(86) PCT No.: PCT/US2021/024169

§ 371 Date: Sep. 26, 2022

Related U.S. Application Data
(60) Provisional application No. 62/994,778, filed on Mar. 25, 2020.

(51) Int. Cl. C07D 213/22

(52) U.S. Cl. CPC C07D 213/22; C07B 2200/13

(57) ABSTRACT

Disclosed herein are compositions comprising air-stable radical [2]catenanes and method of making the same. The [2]catenane comprises a first macrocyclic ring mechanically interlocked with a second macrocyclic ring where each of the first macrocyclic ring and the second macrocyclic ring comprise an alternating cyclic arrangement of a first unsubstituted or substituted 4,4′-bipyridinium (BIPY) subunit, a first unsubstituted or substituted phenylene subunit, a second unsubstituted or substituted BIPY subunit, and a second unsubstituted or substituted phenylene subunit forming a macrocycle.

### CROSS-REFERENCE TO RELATED APPLICATIONS

The present application claims benefit of priority to United States Application Ser. No. 62/994,778, filed Mar. 25, 2020, the contents of which is incorporated by reference in its entirety.

### BACKGROUND OF THE INVENTION

In recent decades, the development of new stable organic radicals has become a topic for extensive investigations^{1,2} because of their unique optical,³ electronic,⁴ and magnetic⁵ properties. Most organic radicals are unstable under ambient conditions and dimerize⁶ quickly-to form new covalent bonds—or become oxidized/reduced under ambient conditions, making their isolation and characterization demanding tasks. In general, there are several common strategies for enhancing the air-stability of organic radicals, such as (i) increasing the steric hindrance around the radical center⁷ in order to prevent dimerization, (ii) introducing electron-withdrawing groups to lower the LUMO energy level⁸ in order to enhance resistance to oxidation by O₂ and H₂O, and (iii) recovering aromaticity⁹, amidst other exmaples¹. However, due to the difficulty in stabilizing organic radicals, new compositions and methods are needed.

### BRIEF SUMMARY OF THE INVENTION

Disclosed herein are compositions comprising air-stable radical [2]catenanes and methods of making the same. One aspect of the invention includes compositions comprising a [2]catenane. The [2]catenane comprises a first ring mechanically interlocked with a second ring or a salt thereof. In some embodiments, the [2]catenane is an air-stable bisradical hexacationic state, an air-stable monoradical heptacationic state, or the mixture of both states. In some embodiment, the first ring, the second ring, or both the first ring and the second ring are mCBPQT. In a particular embodiment, both the first ring and the second ring are mCBPQT. In another embodiment, only one of the first ring and the second ring are mCBPQT.

The compositions described herein may have positive reduction potentials. Suitably, the composition has an E_{red1} greater than +0.50 V versus Ag/AgCl and/or the composition has an E_{red2} greater than +0.25 V versus Ag/AgCl.

The composition described herein may have a near infrared absorption band longer than 1200 nm. Suitably, the near infrared absorption band longer than 1400 nm, 1600 nm, or 1800 nm.

Another aspect of the invention includes crystalline composition. The crystalline composition may comprises any of the compositions described herein and have a molecular packing arranging defined by a triclinic, space group P1⁻(no. 2) or a orthorhombic, space group Pna21 (no. 33). In one embodiment, the composition has a molecular packing arranging defined by the triclinic, space group P1⁻(no. 2) and lattice parameters of a= 14.1 ± 0.1 Å, b = 15.3 ± 0.1 Å, c = 23.3 ± 0.1 Å, α = 84.6 ± 0.1°, β = 82.2 ± 0.1°, and γ = 66.5 ± 0.1°. In another embodiment, the composition has a molecular packing arranging defined by the orthorhombic, space group Pna2₁ (no. 33) and lattice parameters of a = 27.2 ± 0.1 Å, b = 20.4 ± 0.1 Å, and c = 16.7 ± 0.1 Å. Suitably, the composition may comprise six counter anions for every [2]catenane.

Near infrared dyes, memory devices, or energy storage materials may be prepared from any of the compositions described herein.

Yet another aspect of the invention is methods for preparing [2]catenanes. The method may comprise contacting a cationic ring with a cationic guest molecule in the presence of reducing agents, i.e., Cu dust, Zu dust, or CoCp2, etc., thereby reducing the cationic ring and the cationic guest molecule and forming a radical cationic inclusion complex and reacting the guest molecule of the radical cationic inclusion complex with a ring-closing reagent to prepare the [2]catenane or reaching the termini of the guest molecule of the radical cationic inclusion complex with each other to prepare the [2]catenane. In some embodiments, the method further comprises reducing the [2]catenane with reducing agent to prepare a reduced [2]catenane. In some embodiments, the cationic ring is mCBPQT⁴⁺. In one embodiment, the cationic guest molecule may be

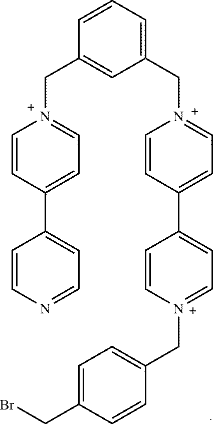

In another embodiment, the cationic guest molecule may be

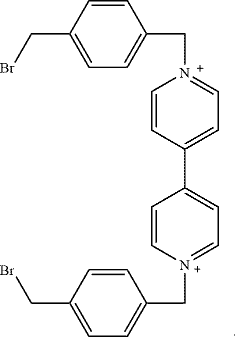

Suitably, the ring-closing reagent may be 4,4′-bipyridine.

These and other aspects of the invention will be described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1C. FIG. 1A) Structural formulas and cavity sizes of CBPQT⁴⁺ and mCBPQT⁴⁺. FIG. 1B) The radical host-guest pairing interactions between mCBPQT^{2(•+)} and the dimethyl viologen radical cation, and the corresponding association constant (Kₐ) in MeCN. FIG. 1C) The reduction potentials, radical stability, and corresponding reference literature of different viologen derivatives-including the newly designed [2]catenanes mHe[2]C⁸⁺ and mHo[2]C⁸⁺— indicating the positive correlation between the reduction potential, and stability of the radicals.

FIGS. 2A-2F. Solid-state structures. FIG. 2A) A side-on view showing the dihedral angle between the BIPY units in mHe[2]C^{2•6+}. FIG. 2B) A top-down view showing the distances and the torsion angles between stacked units in mHe[2]C^{2•6+}. FIG. 2C) A side-on view showing that there are six PF₆⁻ anions surrounding every mHe[2]C^{2•6+}. FIG. 2D) A side-on view showing the dihedral angle between the BIPY units in mHo[2]C^{2•6+}. FIG. 2E) A top-down view showing the distances and the torsion angles between stacked units in mHo[2]C^{2•6+}. FIG. 2F) A side-on view showing that there are six PF₆⁻ anions surrounding every mHo[2]C^{2•6+}.

FIG. 3. Cyclic voltammograms of mHe[2]C·6PF₆ (0.50 mM) and mHo[2]C·6PF₆ (0.50 mM) with the redox potentials marked on all peaks.

FIGS. 4A-4D. Vis/NIR Absorption spectra of the different redox states obtained employing electrochemical reduction at different voltages. FIG. 4A) mHe[2]C^{4(•+)} (-0.50 V) (maxima indicated at 1070 nm); mHe[2]C^{2•6+} (+0.10 V) (maxima indicated at 1450 nm); mHe[2]C^{•7+} (+0.42 V) (maxima indicated at 1800 nm). FIG. 4B) mHo[2]C^{4(•+)} (-0.50 V) (maxima indicated at 1070 nm); mHo[2]C^{2•6+} (+0.10 V) (maxima indicated at 1480 nm); mHo[2]C^{•7+} (+0.50 V) (maxima indicated at 1750 nm). Reference electrode: Ag/AgCl. FIG. 4C) EPR spectra of mHe[2]C^{•7+} (humped line) and mHe[2]C^{2•6+} (flat line). FIG. 4D) EPR spectra of mHo[2]C^{•7+} (humped line) and mHo[2]C^{2•6+} (flat line).

FIGS. 5A-5D. Spin-density distribution of: FIG. 5A) mHe[2]C^{•7+}; FIG. 5B) mHe[2]C^{2•6+}; FIG. 5C) mHo[2]C^{•7+}; FIG. 5D) mHo[2] C^{2•6+}, showing the spin densities are located in the two inner BIPY units for the two [2]catenanes in both mono- and bisradical states.

FIG. 6. Time-dependent Vis/NIR absorption spectra in 30 min intervals (MeCN, optical length: 2 mm) of mHo[2]C^{4(•+)} upon exposing under ambient conditions for 16 h.

FIG. 7. Time-dependent Vis/NIR absorption spectra in 30 min intervals (MeCN, optical length: 2 mm) of mHo[2]C^{4(•+)} upon exposing under ambient conditions for 16 h.

FIG. 8. Time-dependent Vis/NIR absorption spectra (10 mM, MeCN, optical length: 2 mm) of mHe[2]C^{2•6+} upon exposing under ambient conditions.

FIG. 9. Time-dependent Vis/NIR absorption spectra (10 mM, MeCN, optical length: 2 mm) of mHo[2]C^{2•6+} upon exposing under ambient conditions.

### DETAILED DESCRIPTION OF THE INVENTION

Disclosed herein are [2]catenane compositions that allow for air-stable organic radicals. The protection afforded the [2]catenanes by mechanical bonds allow for the remarkable stability of these radicals.

Catenanes are organic compounds having two or more macrocyclic rings connected in the manner of links in a chain, without a covalent bond. Macrocycles are a cyclic macromolecular or a macromolecular cyclic portion of a macromolecule. A molecule of high relative molecular mass, the structure of which essentially comprises the multiple repetition of units derived, actually or conceptually, from molecules of low relative molecular mass. As demonstrated in the Examples that follow, the catenanes are [2]catenanes having two mechanically interlocked rings that result in air-stable radicals, such as bis- and mono-radicals. Suitably, an “air-stable” radical is a radical stable in air for at least 1 hour, 1 day, 1 week, or more. In some embodiments, the air-stable radical is a radical in a bisradical hexacationic state, a monoradical heptacationic state, or a mixture of both states.

The first ring and the second ring of the catenane each comprise an alternating cyclic arrangement of unsubstituted or substituted 4,4′-bipyridinium (BIPY) and phenylene subunits. An exemplary BIPY subunit is a subunit of Formula I,

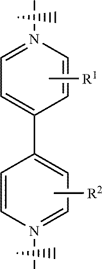

As disclosed in the examples that follow, the BIPY subunit comprises unsubstituted pyridine groups. Derivatives of the unsubstituted BIPY subunit may be prepared and used to form the catenane compositions described herein by replacing any of the hydrogens on either or both of the pyridine rings with one or more substituents. Exemplary substituents R¹ and R² include, but are not limited to, —CH₃, —OH, —NH₂, —SH, —CN, —NO₂, —F, —Cl, —Br, —I moieties. R¹ and R² may be independently selected. In some instances, R¹ and R² are the same. In other instances, R¹ and R² are the different. Because BIPY subunits are threaded through the opposite macrocyclic ring, the substituents on a threaded BIPY subunit must be small enough to allow threading. The second ring may further comprise an additional BIPY subunit that is not threaded through the macrocycle of the opposite ring. Because the additional BIPY subunit is not threaded, substituents such as C₁-C₁₂ alkyl, C₂-C₁₂ alkenyl, C₂-C₁₂ alkynyl, C₁-C₁₂ carboxy, C₁-C₁₂ carbonyl, C₁-C₁₂ aldehyde, or C₁-C₁₂ alkoxy moieties having too much steric bulk to allow threading may also be used for this subunit. In some embodiments, the substituents comprise C₁-C₆ alkyl, C₂-C₆ alkenyl, C₂-C₆ alkynyl, C₁-C₆ carboxy, C₁-C₆ carbonyl, C₁-C₆ aldehyde, or C₁-C₆ alkoxy moieties or C₁-C₄ alkyl, C₂-C₄ alkenyl, C₂-C₄ alkynyl, C₁-C₄ carboxy, C₁-C₄ carbonyl, C₁-C₄ aldehyde, or C₁-C₄ alkoxy moieties.

When a ring comprises two BIPY subunits, the BIPY subunits can be the same or different. Both BIPY units may be unsubstituted, one BIPY subunit may be unsubstituted and the other substituted, or both BIPY subunits may be substituted. When both BIPY subunits are substituted, the BIPY subunits may comprise the same or different substituents.

The BIPY subunits may access a number of different redox states, including as a BIPY²⁺ dication or as a BIPY^{•+} radical cation. Formula I may represent the BIPY²⁺, BIPY^{•+}, or BIPY⁰ redox state depending on context. Moreover, when a ring comprises two BIPY subunits, the subunits may be in the same redox state or different redox states. 4,4′-Bipyridinium radical cations (BIPY^{•+}) tend to form (BIPY^{•+})₂ dimers in a ‘face-to-face’ manner in the solid state as a result of favorable radical-pairing interactions. Conversely, in a dilute solution, (BIPY^{•+})₂ dimers are prone to dissociate because of their low association constants.

The BIPY subunits are linked by phenylene subunits, such as meta-phenylene and/or para-phenylene subunits that may optionally have one or more linkers for joining the BIPY subunits to the phenylene subunits. As disclosed in the examples that follow, the phenylene subunits are unsubstituted. Derivatives of the unsubstituted para-phenylene and/or meta-phenylene subunit may be prepared and used to form the catenane compositions described herein by replacing any of the hydrogens on the phenylene with one or more substituents. Because the phenylene subunits may be threaded through the opposite macrocyclic ring to prepare the catenane, the substituents on a threaded phenylene subunit must be small enough to allow threading. Exemplary substituents include, but are not limited to, alkyl, alkenyl, alkynyl, carboxy, carbonyl, aldehyde, alkoxy, —OH, —NH₂, —SH, —CN, —NO₂, —N₃, —F, —Cl, —Br, —I moieties. In some embodiments, the substituent is -R,-OR, —NH₂, -COOR, —CN, —N₃, —CH═CHR, —NO₂, —F, —Cl, —Br, —I moieties where R is an alkyl, such as a C₁-C₁₂ alkyl, C₁-C₆ alkyl, or C₁-C₄ alkyl.

In some embodiments, the BIPY subunits are linked through a para-xylylene and/or a meta-xylylene subunits where the methylenes of the xylylene are linkers for joining the BIPY subunits to the phenylene subunits. The xylylene subunits may be substituted. Exemplary substituents for the xylylene subunit include, but are not limited to, alkyl, alkenyl, alkynyl, carboxy, carbonyl, aldehyde, alkoxy, —OH, —NH₂, —SH, —CN, —NO₂, —F, —Cl, —Br, —I moieties. In some embodiments, the substituent is -R,-OR, —NH₂, -COOR, —CN, —N₃, —CH═CHR, —NO₂, —F, —Cl, —Br, —I moieties where R is an alkyl, such as a C₁-C₁₂ alkyl, C₁-C₆ alkyl, or C₁-C₄ alkyl.

An exemplary embodiment of one or both of the rings of the [2]catenane is mCBPQT,

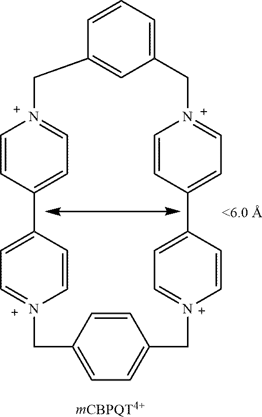

Derivatives may also be prepared by substituting any of the hydrogens on any of the aromatic rings of the BIPY subunit as described above. The m- and p-xylylene subunits may be similarly substituted as described above.

Another exemplary embodiment of one of the rings of the [2]catenane is CBPQT,

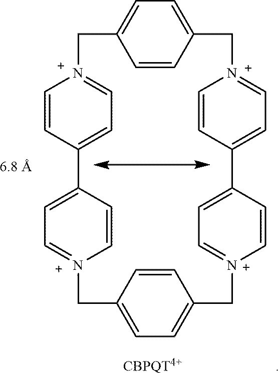

Derivatives may also be prepared by substituting any of the hydrogens on any of the aromatic rings of the BIPY subunit as described above. The p-xylylene subunits may be similarly substituted as described above.

In an embodiment of the invention, the [2]catenane composition comprises two mechanically interlocked mCBPQT. This [2]catenane may be referred to as mHo[2]C.

In an embodiment of the invention, the [2]catenane composition comprises one mechanically interlocked mCBPQT and one mechanically interlocked CBPQT. This [2]catenane may be referred to as mHe[2]C.

The mechanically interlocked compositions may be prepared by providing a radical cationic inclusion complex and preforming a ring-closing reaction. The present methods uses Cu dust to prepare the inclusion complexes, but other reducing agents may also be used. Prior methods for preparing inclusion complexes used Zn dust, however Zn dust must be removed before performing the ring-closing reaction as will over-reduce radical cation, preparing a neutral state, if it remains in the reaction mixture. The advantage of using Cu dust is that it can remain in the reaction mixture with no threat of over-reduction, and can reduce continuously the newly formed radical cations.

The method for preparing a [2]catenane may comprise contracting a cationic ring with a cationic guest molecule in the presence of reducing agent, such as Cu dust. The presence of the Cu dust reduces the cationic ring and the cationic guest molecule into radical cations, respectively. In an embodiment, the cationic ring is mCBPQT⁴⁺ but any of the rings described herein may be employed.

Scheme 1 illustrates two embodiments for preparing the [2]catenane. In a first embodiments, the guest molecule of the radical cationic inclusion complex is reacted with a ring-closing reagent to prepare the [2]catenane. In an alternative embodiment, the termini of the guest molecule of the radical cationic inclusion complex may be reacted with each other to prepare the [2]catenane.

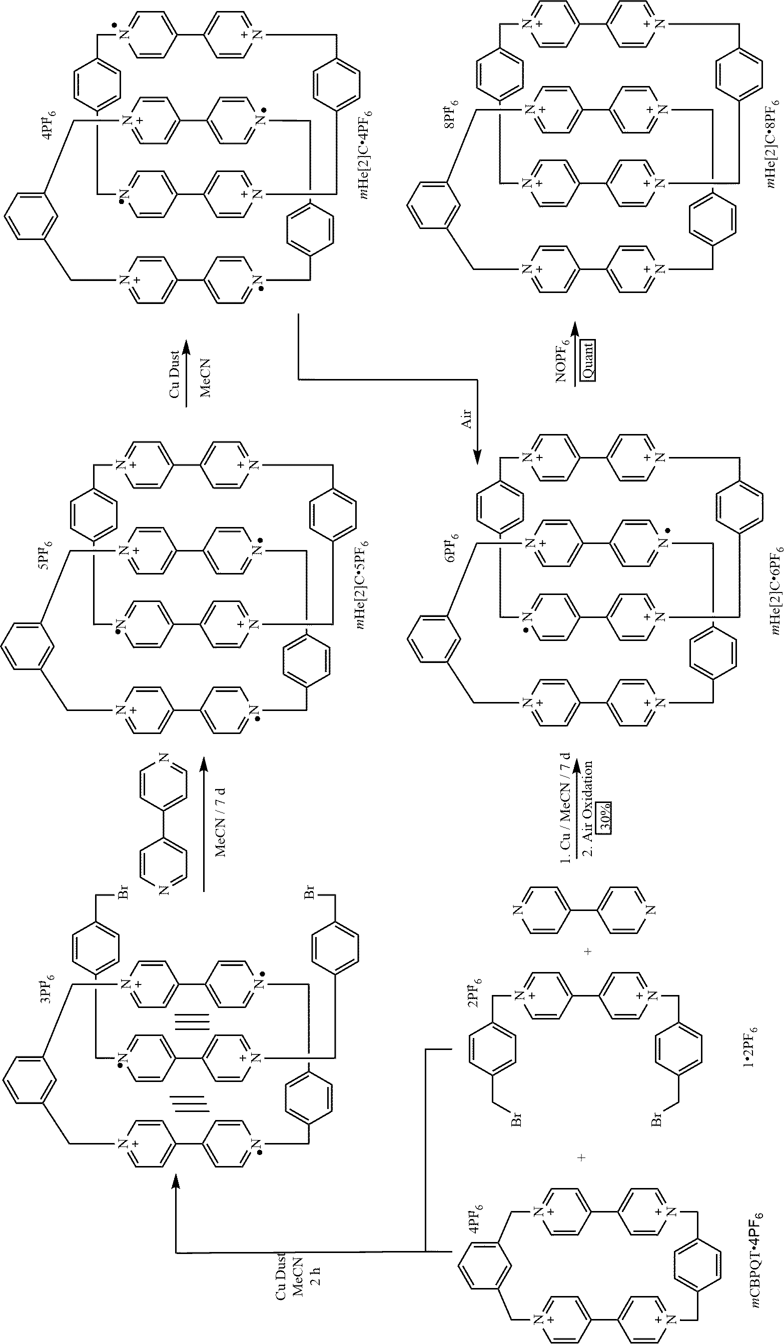

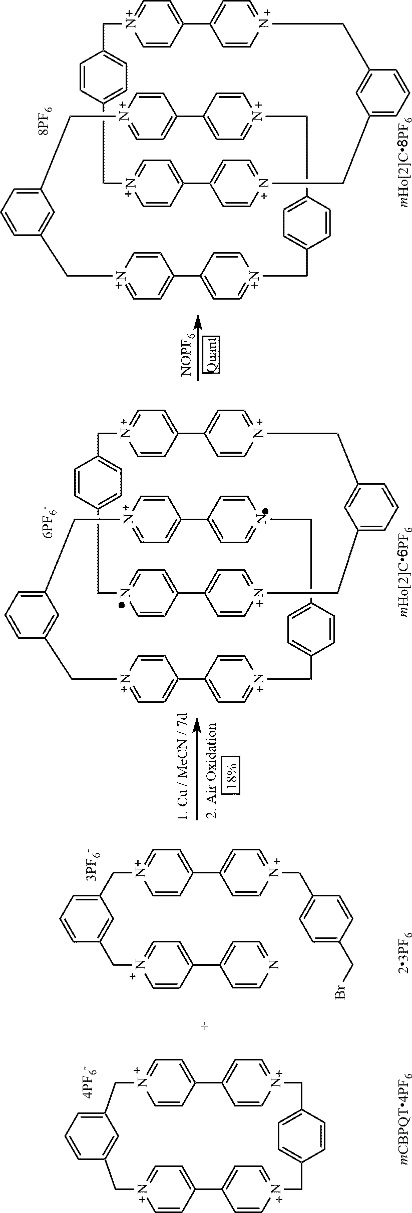

Scheme 1. Synthetic route for the preparation of mHe[2]C·6PF₆, mHo[2]C·6PF₆, mHe[2]C·8PF₆ and mHo[2]C·8PF₆.

As evidenced by two highly charged [2]catenanes, air-stable singlet bisradicals may be prepared and isolated. mHe[2]C•6PF₆ and mHo[2]C•6PF₆ that were synthesized by exploiting radical host-guest templation between BIPY^{•+} derivatives and mCBPQT^{2(•+)}. In contrast to other [2]catenanes that have been isolated as air-stable monoradicals, both mHe[2]C•6PF₆ and mHo[2]C•6PF₆, exist as air-stable singlet bisradicals as evidenced by both X-ray crystallography in the solid state and EPR spectroscopy in solution. Electrochemical studies indicate that the first two reduction peaks of these two [2]catenanes are shifted significantly to more positive potentials, a feature which is responsible for their extraordinary stability in air. The mixed-valence nature of the mono- and bisradical states endows them with unique NIR-absorption properties, e.g., NIR absorption bands for the mono- and bisradical states observed at ∼1800 and ∼1450 nm, respectively. These [2]catenanes are useful in applications that include NIR photothermal conversion, UV/Vis/NIR multiple-state electrochromic materials, and multiple-state memory devices. Our findings highlight the principle of “mechanical-bond-induced-stabilization” as an efficient strategy for designing persistent organic radicals.

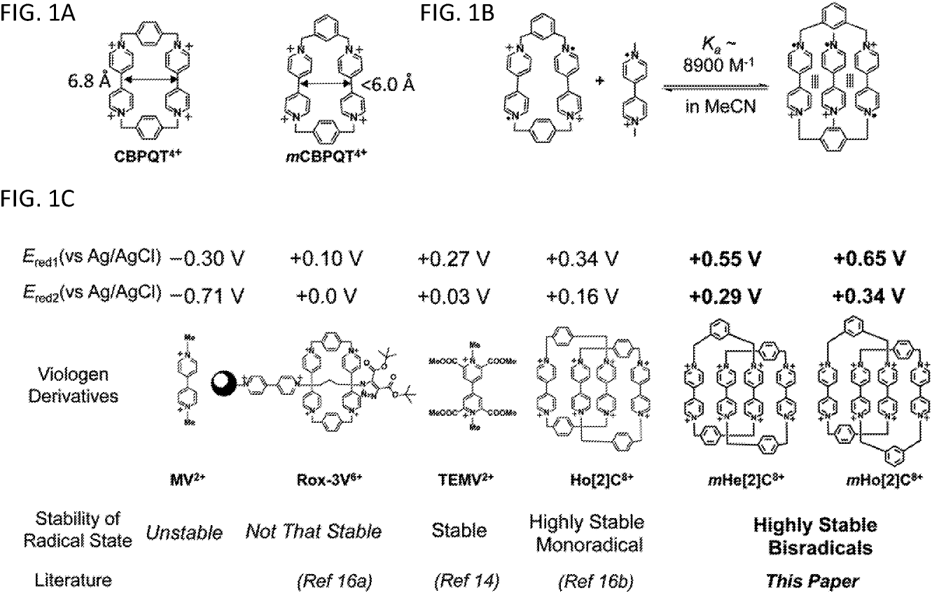

N,N′-Disubstituted-4,4′-bipyridinium dications (BIPY²⁺), also known as viologens¹⁰, are electron acceptors that can undergo two sequential and reversible one-electron reductions with half-wave potentials of -0.30 and -0.71 V (versus Ag/AgCl in MeCN). The bipyridinium radical cation (BIPY^{•+}), which is generated from the one-electron reduction of BIPY²⁺, is a well-known thermally stable radical species in an inert atmosphere, and can undergo (noncovalent) π-dimerization¹¹ on account of radical-radical interactions; such interactions have been exploited intensively in supramolecular chemistry¹² and mechanostereochemistry¹³. Although, BIPY^{•+} cannot undergo σ-dimerization to form a covalent bond, it is unstable when exposed to air because the BIPY²⁺/BIPY^{•+} reduction potential (-0.30 V versus Ag/AgCl) is not sufficiently positive for the radicals to resist aerobic oxidation. One way to tune the reduction potential of viologens towards more positive values involves introducing electron-withdrawing substituents onto viologen derivatives that makes them more electron-deficient, as exemplified (FIGS. 1A-1C) by tetramethyl esters functionalized¹⁴ dimethyl viologen, TEMV²⁺. The first reduction potential of TEMV²⁺ is shifted to around +0.27 V versus Ag/AgCl relative to that (-0.30 V) of the original dimethyl viologen radical cation (MV^{•+}), and so the air-stability of the TEMV^{•+} radical cation turned out to be improved¹⁴ significantly.

Cyclobis(paraquat-p-phenylene) bisradical dication CBPQT^{2(•+)}, shown in FIG. 1A, can accommodate a BIPY^{•+} radical cation to form the trisradical tricationic complex BIPY^{•+}⊂CBPQT^{2(•+)} in MeCN.¹⁵ Using this complex as a templating motif, we have synthesized¹⁶ a series of highly positively charged mechanically interlocked molecules (MIMs), i.e., Rox-3V⁶⁺ and Ho[2]C⁸⁺ shown in FIG. 1C. We found that the reduction potentials of these MIMs are shifted to significantly more positive values when the positively charged components in the MIMs are forced into nano-confinement as a result of mechanical bonding which stabilizes the radical states under ambient conditions. This property is especially evident in the homo[2]catenane, Ho[2]C⁸⁺, in which the four repulsive viologen units are obliged to stack with π-overlap in a very small volume (<1.25 nm³), a situation that brings about a strong tendency for Ho[2]C⁸⁺ to accept electrons, resulting in the stabilization of the monoradical Ho[2]C^{•7+} state under ambient conditions.

mCBPQT^{2(•+)} also associates with MV^{•+} in MeCN, despite its cavity being significantly smaller (FIGS. 1A and 1B) than that present in CBPQT^{2(•+)}. Since the mCBPQT⁴⁺ cavity is smaller¹⁷ than that of CBPQT⁴⁺, the four electrostatically repulsive viologen units stack in an even more compact manner than those present in Ho[2]C⁸⁺. A bolded descriptor may denote a compound, be it free or complexed, and an unbolded descriptor refers to either (i) a component within a molecule or (ii) a component part of a mechanically interlocked molecule. Consequently, both of the two inner BIPY²⁺ units in mHe[2]C⁸⁺ and mHo[2]C⁸⁺ are expected to be more easily reduced than those in Ho[2]C⁸⁺. If the second reduction potentials of mHe[2]C⁸⁺ and mHo[2]C⁸⁺ are shifted positively to values that make aerobic oxidation difficult, then the bisradical forms— namely, mHe[2]C^{2•6+} and mHo[2]C^{2•6+}—will be stable under ambient conditions. Since the first reduction potential of TEMV²⁺ (FIGS. 1A-1C) is around +0.27 V versus Ag/AgCl, we believe that, if the second reduction potentials of the new [2]catenanes are more positive than +0.27 V, then their bisradical states will have comparable or even better air-stability than TEMV^{•+}. The mixed-valence nature of the mono- and bisradical states is responsible for their unique NIR absorption properties. These new [2]catenanes are useful in a variety of ways, such as in NIR-II photothermal conversion,²² UV/Vis/NIR multistate electrochromic materials,²³ and multistate information storage/memory devices²⁴.

### Definitions

As used herein, an asterick “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C1-C12 alkyl, C1-C10-alkyl, and C1-C6-alkyl, respectively.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like

The term “heteroalkyl” as used herein refers to an “alkyl” group in which at least one carbon atom has been replaced with a heteroatom (e.g., an O, N, or S atom). One type of heteroalkyl group is an “alkoxyl” group

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number oring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, -C(O)alkyl, -CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3-to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O—alkyl, —O—alkenyl, —O—alkynyl, and the like.

An “epoxide” is a cyclic ether with a three-atom ring typically include two carbon atoms and whose shape approximates an isosceles triangle. Epoxides can be formed by oxidation of a double bound where the carbon atoms of the double bond form an epoxide with an oxygen atom.

The term “carbonyl” as used herein refers to the radical —C(O)—.

The term “carboxamido” as used herein refers to the radical —C(O)NRR′, where R and R′ may be the same or different. Rand R′ may be independently alkyl, aryl, arylalkyl, cycloalkyl, formyl, haloalkyl, heteroaryl, or heterocyclyl.

The term “carboxy” as used herein refers to the radical -COOH or its corresponding salts, e.g. —COONa, etc.

The term “amide” or “amido” as used herein refers to a radical of the form —R¹C(O)N(R²)—, —R¹C(O)N(R²)R³—, —C(O)NR²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### General Methods

All reagents were purchased from commercial suppliers and used without further purification. Compounds 1•2PF₆, CBPQT•4PF₆, mCBPQT•4PF₆ were prepared^{S1} according to literature procedures. Thin layer chromatography (TLC) was performed on silica gel 60 F254 (E. Merck). Column chromatography was carried out on silica gel 60F (Merck 9385, 0.040-0.063 mm). C-18 Columns were used for analytical and preparative reverse-phase high-performance liquid-chromatography (RP-HPLC) on Agilent 1260 infinity LC equipped with Agilent 6120 LC/MS electrospray system and Shimadzu Prominence LC-8a instruments, respectively, eluted with H₂O/MeCN (0.1 % v/v TFA) and monitored using a UV detector (λ = 360 nm). UV/Vis Spectra were recorded at room temperature on a Shimadzu UV-3600 spectrophotometer. Nuclear magnetic resonance (NMR) spectra are recorded on Agilent DD2 500 as well as on Bruker Avance III 400 and 500 spectrometers, with working frequencies of 400 and 500 MHz for ¹H, as well as 100 and 125 MHz for ¹³ C nuclei, respectively. Chemical shifts were reported in ppm relative to the signals corresponding to the residual non-deuterated solvents (CD₃CN: δ_{H} =1.94 ppm and δ_{C} = 118.26 ppm for ¹³CN). High-resolution mass spectra (HR-ESI) were measured on a Finnigan LCQ iontrap mass spectrometer. Electron paramagnetic resonance (EPR) measurements at X-band (9.5 GHz) were performed with a Bruker Elexsys E580, equipped with a 4122SHQE resonator. All samples were prepared in an Argon-filled atmosphere. Scans were performed with magnetic field modulation amplitude of 1 G and non-saturating microwave power between 0.4 and 0.6 mW. Samples were contained in quartz tubes with I.D. 1.50 mm and O.D. 1.80 mm and sealed with a clear ridged UV curin epoxy (IllumaBond 60-7160RCL) and used immediately after preparation. Cyclic voltammetry (CV) and differential pulse voltammetry (DPV) experiments were carried out at room temperature in Argon-purged MeCN solutions with a Gamry Multipurpose instrument (Reference 600) interfaced to a PC. CV Experiments were performed using a glassy carbon working electrode (0.071 cm²). The electrode surface was polished routinely with 0.05 µm alumina-water slurry on a felt surface immediately before use. The counter electrode was a Pt coil and the reference electrode was Ag/AgCl electrode. The concentration of the supporting electrolyte tetrabutylammonium hexafluorophosphate (NH₄PF₆) was 0.1 M.

### Synthesis

The highly stable bisradical [2]catenanes, mHe[2]C•6PF₆ and mHo[2]C·6PF₆, were synthesized by modifying the previously reported procedure^{16b} for the preparation of Ho[2]C•7PF₆ The mCBPQT•4PF₆ host and the guest molecule 1•2PF₆ were reduced (Scheme 1) with an excess of Cu dust in MeCN in a N₂-filled glovebox for 2 h, producing the trisradical tricationic inclusion complex 1^{•+}⊂mCBPQT^{2(•+)}. 4,4′-Bipyridine was then added to this solution so as to react with 1•2PF₆ and give mHe[2]C^{3•5+} as the ring-closure product, which was then reduced again by the Cu dust to give mHe[2]C^{4(•+)}. The reaction mixture was stirred at room temperature under N₂ for 1 week, after which it was exposed to air. Purification by reverse phase column chromatography, followed by counterion exchange, and recrystallization (see Section B) afforded mHe[2]C•6PF₆ in 30% yield.

In a similar manner, mHo[2]C•6PF₆ was obtained in 19% yield by reacting mCBPQT•4PF₆ with 2•3PF₆ using the same protocol. The lower yield of mHo[2]C•6PF₆ can be attributed to the smaller cavity of mCBPQT^{2(+•)} compared to that of CBPQT^{2(+•)}, which renders the ring-closure step for mHo[2]C•6PF₆ more difficult than that for mHe[2]C•6PF₆. High resolution electrospray ionization mass spectrometry (ESI-MS) confirmed that both catenanes possess the same molecular formula, i.e., C₇₂H₆₄F₃₆N₈P₆.

¹H NMR Spectra have been recorded for both catenanes in their fully oxidized states— namely, mHe[2]C·8PF₆ and mHo[2]C·8PF₆ —which were obtained by oxidizing the as-synthesized catenanes with an excess of NOPF₆. Because of their lower symmetries, both mHe[2]C·8PF₆ and mHo[2]C·8PF₆ display much more complicated ¹H NMR spectra than that observed for Ho[2]C·8PF₆. The characteristic signals of these [2]catenanes correspond to the proton resonances of the innermost BIPY²⁺ units, which are strongly shielded and consequently shifted dramatically upfield, into the 4-5 ppm region. Notably, the eight resonances for the innermost protons are separated into two sets of signals for mHe[2]C·8PF₆ (two protons resonate at ∼5.10 ppm and six protons resonate at ∼4.25 ppm), while these same eight proton resonances in the spectrum of mHo[2]C·8PF₆ are separated into four sets of signals at 5.29, 4.98, 4.38, and 4.07 ppm. These observations can be attributed to the asymmetric cavities of the mCBPQT⁴⁺ component ring(s). The encapsulated BIPY²⁺ unit(s) are obliged to reside closer to the p-xylylene linker end than the m-xylylene linker end in order to attenuate Coulombic repulsions as much as possible. As a consequence, the innermost protons on the BIPY²⁺ units of mCBPQT⁴⁺ experience different extents of shielding, leading to well separated chemical shifts. The remaining proton resonances in the spectra of mHe[2]C·8PF₆ and mHo[2]C·8PF₆ are also more complicated for similar reasons.

The previously reported protocol in Reference 16b uses Zn dust as the reducing agent to generate rapidly the trisradical tricationic complexes. Zn dust, however, must be removed from the reaction mixture before BIPY is added to the solution so as to react with the encapsulated xylylene dibromide. Zn will over-reduce the viologen radical cation to give its neutral state if it remains in the reaction mixture. Once the substitution is over, however, the newly formed BIPY²⁺ cannot be reduced because of the absence of reducing reagents in the solution. Hence, there will be fast electron exchange between the newly formed BIPY²⁺ units and the trisradical tricationic complexes, a situation which will reduce the formation of the trisradical tricationic complexes and therefore decrease the catenation yield. The advantage of using Cu dust is that it can remain in the reaction mixture with no threat of over-reduction, and can reduce continuously the newly formed BIPY²⁺ units to radical cations. Synthetic Protocols

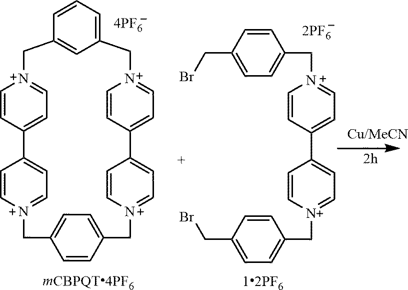

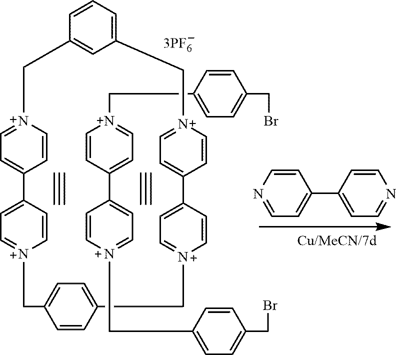

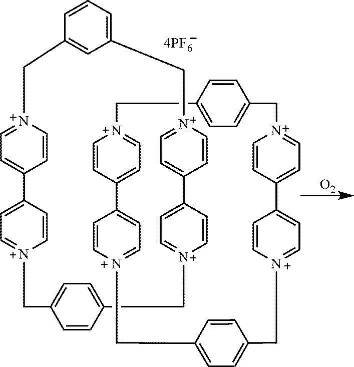

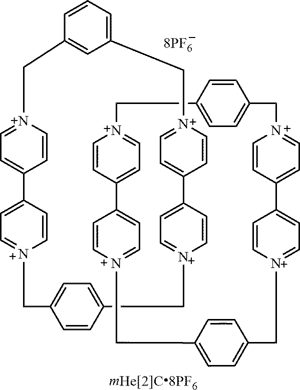

mHeC•6PF₆: mCBPQT•4PF₆ (330 mg, 0.30 mmol) and 1•2PF₆ (270 mg, 0.34 mmol) were dissolved in degassed MeCN (60 mL) in a 100-mL round-bottomed flask in a N₂-filled glovebox. An excess of Cu dust (∼100 mg) was added with stirring to this solution. After 2 h, the solution turned from colorless to a deep purple color, an observation which is indicative of the formation of the trisradical tricationic complex. Then 4,4′-bipyridine (54 mg, 0.34 mmol) was added to it, and the resulting mixture was allowed to stand for 7 days at room temperature. Cu Dust was removed by filtration and the solvent was evaporated off under vacuum. The crude product was purified using reversed-phase flash chromatography (C₁₈: H₂O/MeCN 0.1% TFA 0-100%), followed using anion exchange from TFA⁻ to PF₆⁻ by treating the aqueous fractions with an excess of NH₄PF₆, resulting in a white precipitate which was collected by centrifugation and washed with H₂O several times before being dried in vacuo to afford mHe[2]C•6PF₆ as a dark purple solid (170 mg, 30%). For NMR spectroscopic characterization, mHe[2]C•6PF₆ (2 mg) was oxidized to mHe[2]C•8PF₆ by the addition of an excess (1 mg) of NO•PF₆. ¹H NMR (500 MHz, CD₃CN, 298 K) of mHe[2]C•8PF₆: δ 9.03 (d, J= 6.6, 6 H), 8.99 (d, J= 7.0 Hz, 2H), 8.87 (d, J= 6.6 Hz, 2 H), 8.83 (d, J= 6.6 Hz, 2 H), 8.72 (d, J= 6.0 Hz, 2 H), 8.40 (s, br, 1 H), 8.34-8.32 (m, 4 H), 8.17 (d, J = 7.0 Hz, 2 H), 8.14 (s, br, 9 H), 8.06 (s, br, 2 H), 7.85 (d, J = 6.5 Hz, 2 H), 7.79 (d, J = 6.5 Hz, 4 H), 7.74 (d, J = 6.5 Hz, 2 H), 6.17-6.13 (m, 6 H), 6.00-5.96 (m, 10H), 5.09 (s, br, 2 H), 4.28-4.22 (m, 6 H). ¹³ C NMR (125 MHz, CD₃CN, 298 K) of mHe[2]C•8PF₆: δ 149.0, 148.8, 148.3, 148.0, 147.7, 147.3, 146.9, 146.6, 143.4, 143.2, 142.8, 140.4, 140.0, 138.8, 138.2, 136.9, 136.8, 135.8, 135.1, 134.7, 133.0, 132.9, 132.5, 132.0, 128.2, 128.0, 127.7, 122.9, 122.9, 122.5, 67.5, 67.2, 67.0, 65.6, 65.5, 65.4. ESI-HRMS for mHe[2]C•6PF₆; Calcd for C₇₂H₆₄F₃₆N₈P₆: m/z = 810.1905 [M- 2PF₆]²⁺; found: 810.1889.

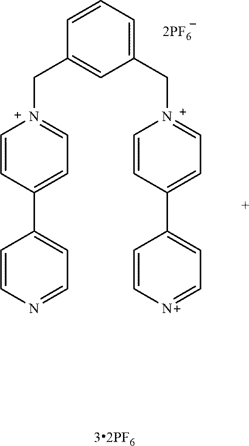

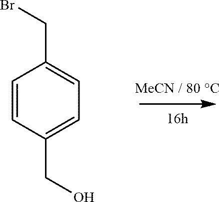

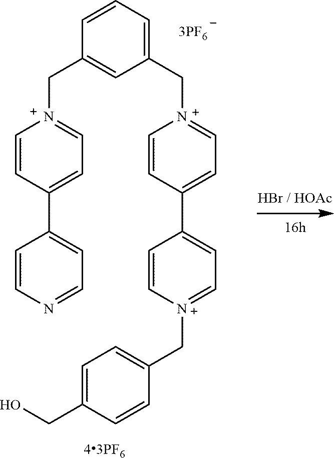

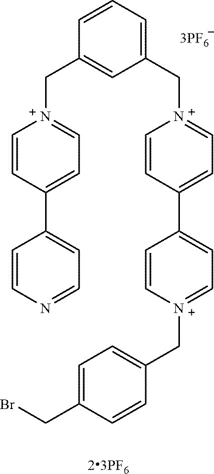

4•3PF₆: 3•2PF₆ (350 mg, 0.50 mmol) and (4-(bromomethyl)phenyl)methanol (120 mg, 0.60 mmol) were dissolved in degassed MeCN (60 mL) in a 100-mL round-bottomed flask. The solution was heated to 80° C. for 16 h with stirring. After cooling down to room temp, excess of TBAC1 was added to the solution. The solids were collected by filtration, and the crude product was purified by reversed-phase flash chromatography (C₁₈: H₂O/MeCN 0.1% TFA 0-100%), followed by anion exchange from TFA⁻ to PF₆⁻ by treating the aqueous fractions with an excess of NH₄PF₆, resulting in a white precipitate which was collected by centrifugation and washed with H₂O several times before being dried in vacuo to afford 4•3PF₆ as an off-white solid (211 mg, 45%). ¹H NMR (500 MHz, CD₃CN, 298 K): δ 9.00-8.94 (m, 8 H), 8.42-8.37 (m, 8 H), 7.63-7.59 (m, 4 H), 7.59 (s, 4 H), 5.87-5.85 (m, 4 H), 5.83 (s, 2 H), 4.65 (s, 2 H). ¹³ C NMR (125 MHz, CD₃CN, 298 K) δ 151.9, 151.4, 151.0, 146.5, 146.4, 145.2, 143.9, 134.7, 132.0, 131.6, 131.6, 131.3, 130.2, 128.44, 128.42, 128.36, 128.32, 127.4, 100.8, 65.4, 64.9, 64.8.

2•3PF₆: 4•2PF₆ (160 mg, 0.83 mmol) was dissolved in HBr (33% in HOAc) (10 mL) in a 25-mL vial. The solution stirred under rt for 16 before the all the solvents were removed in vacuo. The residue was dissolved in water (10 mL), and excess NH₄PF₆ were added to the solution. The precipitate was collected by filtration, washed with H₂O for several times before being dried in vacuo to afford 2•3PF₆ as an off-white solid (153 mg, 92%). ¹H NMR (500 MHz, CD₃CN, 298 K): δ 9.00-8.93 (m, 8 H), 8.41-8.38 (m, 8 H), 7.64-7.50 (m, 8 H), 5.85-5.84 (m, 6 H), 4.654 (s, 2 H). ¹³ C NMR (125 MHz, CD₃CN, 298 K) δ 151.9, 151.3, 151.1, 146.5, 146.5, 145.2, 143.9, 134.7, 132.0, 131.6, 131.6, 131.3, 130.2, 128.46, 128.40, 128.35, 128.2, 127.4, 100.8, 65.4, 65.0, 63.6.

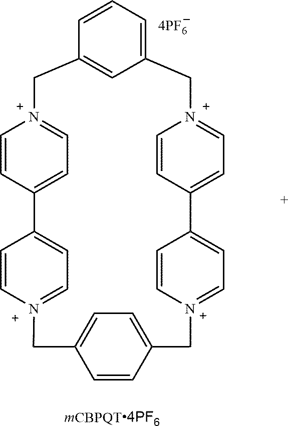

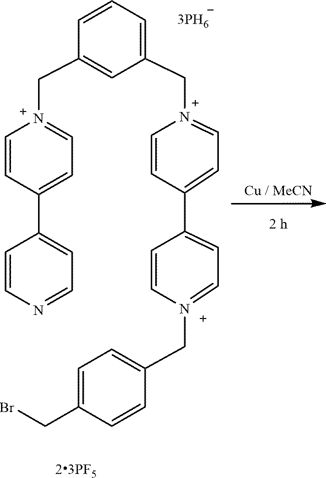

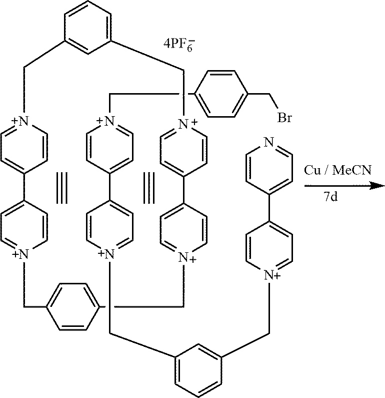

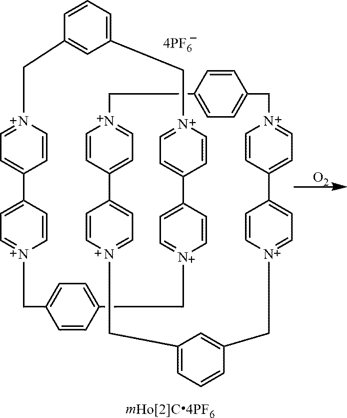

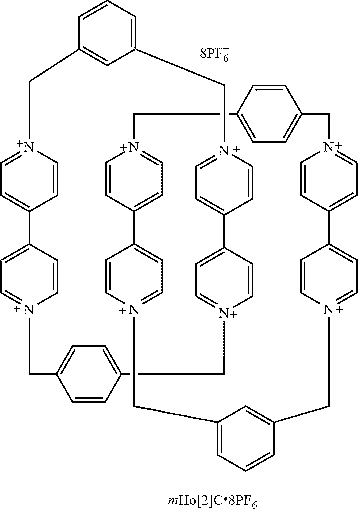

mHoC•6PF₆: Following a procedure similar to that described for the synthesis of mHe [2]C•6PF₆, the reaction of a mixture composed of mCBPQT•4PF₆ (220 mg, 0.20 mmol), 2•3PF₆ (142 mg, 0.17 mmol) afforded mHo[2]C•6PF₆ as a dark purple solid (69 mg, 18%). For NMR spectroscopic characterization, mHo[2]C•6PF₆ (2 mg) was oxidized to mHo[2]C•8PF₆ by the addition of an excess (1 mg) of NO•PF₆. ¹H NMR (500 MHz, CD₃CN, 298 K) of mHo[2]C·8PF₆: δ 9.09 (d, J= 8.0 Hz, 2 H), 9.03-9.01 (m, 4 H), 8.98 (d, J= 7.5 Hz, 2 H), 8.82 (d, J= 6.5 Hz, 2 H), 8.76 (d, J= 6.5 Hz, 8 H), 8.42-8.32 (m, 18 H), 7.87-7.85 (m, 4 H), 7.77-7.75 (m, 4 H), 7.65-7.63 (m, 2 H), 6.20-5.97 (m, 16 H), 5.29 (s, br, 2 H), 4.98 (s, br, 2 H), 4.38 (s, br, 2 H), 4.07 (s, br, 2 H). ¹³C NMR (125 MHz, CD₃CN, 298 K) of SC•8PF₆: δ 148.3, 147.7, 147.5, 147.0, 146.6, 146.3, 143.3, 142.4, 142.2, 140.0, 138.6, 136.6, 136.3, 135.2, 134.5, 132.8, 131.9, 131.4, 131.0, 128.1, 127.9, 127.6, 127.3, 67.1, 66.4, 65.4, 65.3. ESI-HRMS for mHo[2]C•6PF₆; Calcd for C₇₂H₆₄F₃₆N₈P₆: m/z = 810.1905 [M- 2PF₆]²⁺; found: 810.1895.

### X-Ray Crystallography

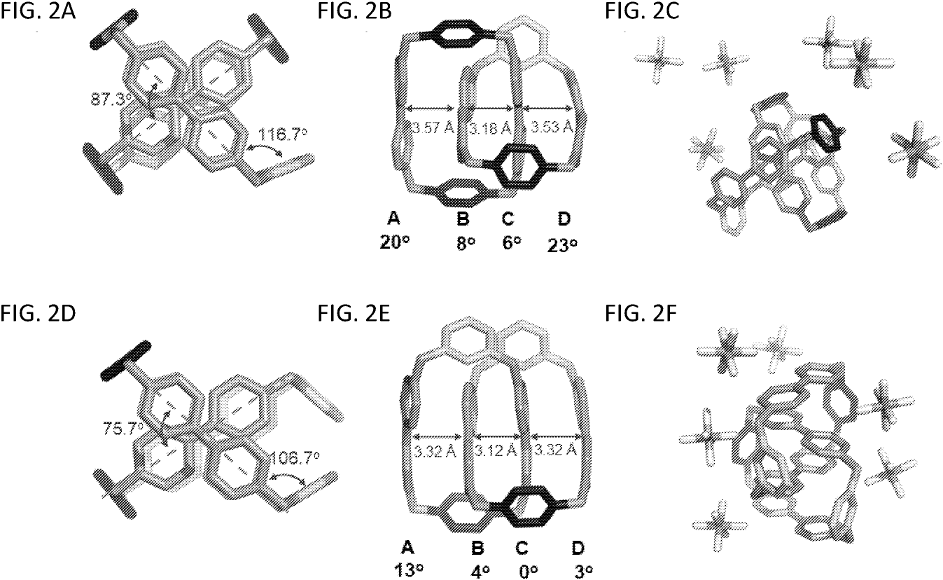

Single crystals of the two catenanes were grown under ambient conditions by slowly evaporating Et₂O into 1.0 mM MeCN solutions over a week which affords dark red crystals suitable for X-ray crystallographic analysis. The solid-state structures show (FIGS. 2A-2F) that both compounds crystallize with six PF₆⁻ counterions around the catenanes, an observation which confirms their bisradical hexacationic states under ambient conditions. The torsional angles (FIG. 2B) of 20° and 23° for the A and D units, respectively, in mHe[2]C^{2•6+} are typical of dicationic BIPY²⁺ units, and indicate that the unpaired electrons are not located on the A and D units. By contrast, units B and C show (FIG. 2B) much smaller torsion angles-8° for unit B and 6° for unit C— indicating that the unpaired electrons are most likely to be located between these units. Moreover, the centroid-to-centroid distance (3.18 Å) between units B and C is significantly shorter than that observed between (3.57 Å) units A and B or between (3.53 Å) units C and D. The value of the distance (3.18 Å) between units B and C is a typically associated with radical-radical interactions, an observation that supports the existence of radical-radical pairing interactions between units B and C. In the case of mHo[2]C^{2•6+}, however, all four units (A, B, C, and D) present (FIG. 2E) near planar conformations with somewhat smaller (<13°) torsion angles, as well as shorter (<3.57 Å) distances between adjacent units when compared to those in mHe[2]C^{2•6+}. These observations can be explained by the small cavities of the two mCBPQT rings, which force the four viologen units to stack more tightly in mHo[2]C^{2•6+} than in mHe[2]C^{2•6+}, thereby leading to flatter viologen-unit conformations and shorter separations. Nevertheless, the torsion angles of the two inner units, B (4°) and C (0°), are still smaller than those of the outer units, A (13°) and D (3°), while the centroid-to-centroid distance (3.12 Å) between units B and C is also smaller than that between (3.39 Å) units A and B or between (3.34 Å) units C and D. Therefore, we conclude that the two spins in mHo[2]C^{2•6+} are also mainly located on units B and C. 1) mHe[2]C•6PF₆
a) Methods. Single crystals of mHe[2]C•6PF₆ were grown on the bench-top by slow vapor diffusion of ⁱPr₂O into a 1.0 mM solution in MeCN over the course of a week. A suitable crystal was selected and mounted in inert oil and transferred to the cold gas stream of a Bruker Kappa Apex2 diffractometer. The crystal was kept at 100 K during data collection. Using Olex2^{S2}, the structure was solved with the XM^{S3} structure solution program using dual space and refined with the XL^{S4} refinement package using least squares minimization.
b) Crystal data. Triclinic, space group P1̅ (no. 2), a= 14.1120(2), b = 15.2780(2), c = 23.3050(3) Å, α = 84.5780(10), β = 82.1550(10), γ = 66.5170(10)°, V= 4560.90(11) Å³, Z = 2, T= 100.01(10) K, µ(CuK_{α}) = 2.200 mm⁻¹, D_{calc}= 1.481 g/mm³, 58617 reflections measured (3.83 ≤ 2Θ ≤ 146.334), 17719 unique (Rᵢₙₜ = 0.0557, R_{sigma} = 0.0565) which were used in all calculations. The final R₁ was 0.0960 (I > 2σ(I)) and wR₂ was 0.3126 (all data).
c) Refinement details. The enhanced rigid-bond restraint (SHELX keyword RIGU) was applied on the disordered PF₆⁻ anions. Distance restraints were also imposed on the disordered anions.
d) Solvent treatment details. The solvent masking procedure as implemented in Olex2^{S2} was used to remove the electronic contribution of solvent molecules from the refinement. As the exact solvent content is not known, only the atoms used in the refinement model are reported in the formula here. 2) mHo[2]C•6PF₆
a) Methods. Single crystals of mHo[2]C•6PF₆ were grown on the bench-top by slow vapor diffusion of ⁱPr₂O into a 1.0 mM solution in MeCN over the course of a week. A suitable single crystal was selected and mounted in inert oil and transferred to the cold gas stream of a Bruker APEX-II CCD diffractometer. The crystal was kept at 100 K during data collection. Using Olex2^{S2}, the structure was solved with the XM^{S3} structure solution program using dual space and refined with the XL^{S4} refinement package using least squares minimization.
b) Crystal data. Orthorhombic, space group Pna2₁ (no. 33), α = 27.2161(4), b = 20.3618(3), c = 16.7417(3) Å, V = 9277.7(3) Å³, Z= 4, T= 100.01(10) K, µ(CuK_{α}) = 2.163 mm⁻¹, D_{calc} = 1.456 g/mm³, 65212 reflections measured (5.42 ≤ 2Θ ≤ 154.996), 17763 unique (Rᵢₙₜ = 0.0527, R_{sigma} = 0.0496) which were used in all calculations. The final R₁ was 0.0609 (I > 2σ(I)) and wR₂ was 0.1712 (all data).
c) Refinement details. Distance a restraints were imposed on the disordered PF₆⁻ anions. The enhanced rigid-bond restraint (SHELX keyword RIGU) was applied to the disordered PF₆⁻ anions.
d) Solvent treatment details. The solvent masking procedure as implemented in Olex2 was used to remove the electronic contribution of solvent molecules from the refinement. As the exact solvent content is not known, only the atoms used in the refinement model are reported in the formula here. Total solvent accessible volume / cell = 3555.8 Å³ [32.0%] Total electron count / cell = 429.5

### Electrochemistry

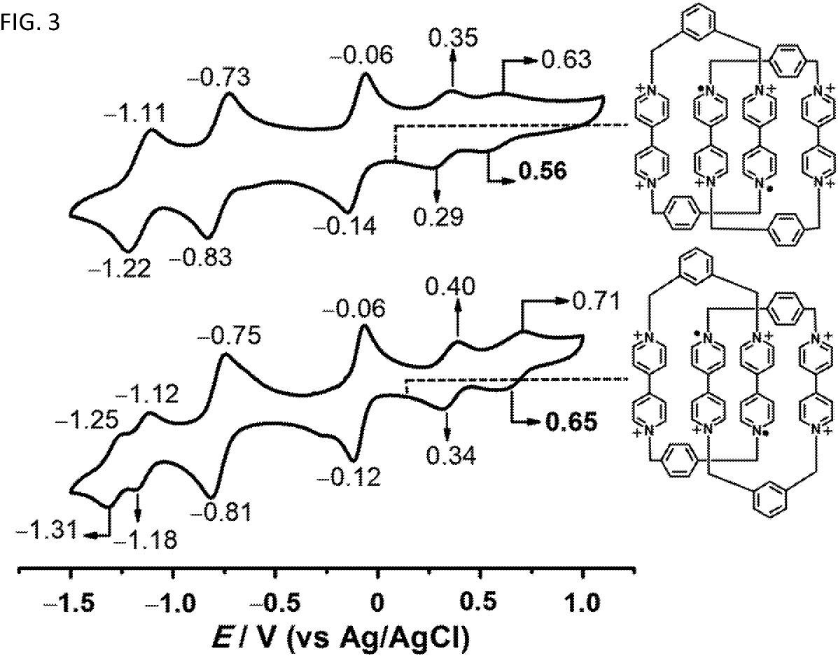

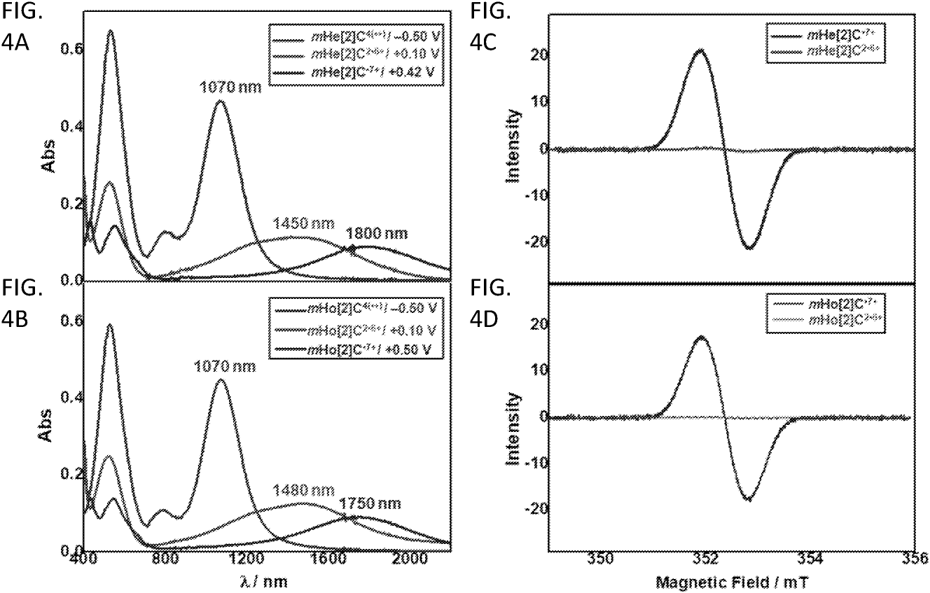

Since the stabilities of viologen radicals in air are mainly determined by their potentials, the redox properties of mHe[2]C·6PF₆ and mHo[2]C·6PF₆ were investigated by cyclic voltammetry (CV). The CV curve for mHe[2]C·6PF₆ exhibits (FIG. 3) five reversible waves corresponding to six discrete accessible redox states, which are similar to those observed previously for Ho[2]C·7PF₆. On the other hand, mHo[2]C·6PF₆ displays (FIG. 3) six reversible waves because of further splitting of its last reduction peaks. Notably, the first two reduction peaks of both mHe[2]C⁸⁺ (E_{red1} = +0.56 V and E_{red2} = +0.29 V versus Ag/AgCl) and mHo[2]C⁸⁺ (E_{red1} = +0.65 V and E_{red2} = +0.34 V versus Ag/AgCl) are significantly positively shifted compared to those previously observed for Ho[2]C⁸⁺ (E_{red1} = +0.34 V and E_{red2} = +0.16 V versus Ag/AgCl). The LUMO energy levels of mHe[2]C⁸⁺ and mHo[2]C⁸⁺ which are calculated to be -5.17 and -5.27 eV, respectively, both are much lower than those for tetracyanoquinodimethane (E_{LUMO} = -4.84 eV) and some other^{2d,8} strong electron acceptors. This observation supports our hypothesis that decreasing the cavity sizes of the component-ring in these highly positively charged catenanes enhances their electron-accepting abilities. In addition, the last reduction peaks of mHe[2]C⁸⁺ (E_{red5} = -1.31 V versus Ag/AgCl) and mHo[2]C⁸⁺ (E_{red5} = -1.22 V versus Ag/AgCl) are both negatively shifted compared to the last reduction peak of Ho[2]C⁸⁺ (E_{red5} = -1.06 V versus Ag/AgCl). Taken all together, these observations demonstrate clearly that subtle differences in the cyclophane linkers influence significantly the redox behavior of these highly positively charged [2]catenanes.

Significantly, the potentials of the second reduction peaks of mHe[2]C⁸⁺ (E_{red2} = +0.29 V) and mHo[2]C⁸⁺ (E_{red2} = +0.34 V) are comparable to the first reduction potentials of TEMV²⁺ (E_{red1} = +0.29 V, FIG. 1C) and Ho[2]C⁸⁺ (E_{red1} = +0.35 V, FIG. 1C). Accordingly, the bisradical states of mHe[2]C⁸⁺ and mHo[2]C⁸⁺ are very like to exhibit similar stabilities in air as TEMV^{•+} and Ho[2]C^{•7+}.

### UV-Vis-NIR Spectroscopy

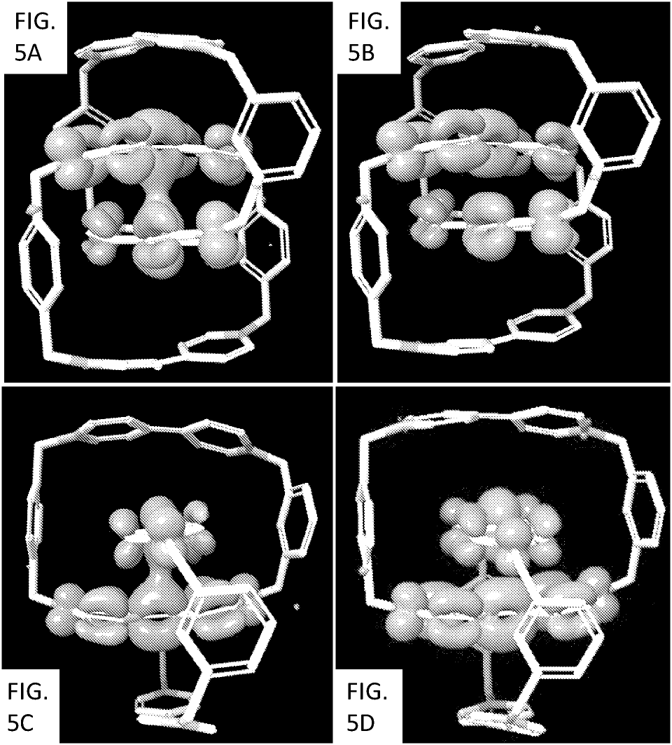

In order to gain additional insight into their electronic properties, we recorded the UV-Vis-NIR spectra of the two [2]catenanes in their various electrochemically generated redox states at different potentials. The CV traces (FIG. 3) reveal that MeCN solutions of mHe[2]C·6PF₆ (+0.80, +0.42, +0.10, and -0.50 V) and mHo[2]C·6PF₆ (+0.80, +0.50, +0.10 and -0.50 V) require different potentials in order to generate the corresponding redox states (8+, 7+, 6+, and 4+). UV-Vis-NIR Spectra of the MeCN solutions of the 7+ (monoradical), 6+ (bisradical), and 4+ (tetraradical) redox states were recorded. See FIGS. 4A-4B. Notably, the mono- and bisradical states of both [2]catenanes exhibit NIR absorption bands around 1800 nm and 1440 nm, respectively, and both of these absorptions are significantly red-shifted compared^{12c} to those of BIPY^{•+} (around 600 nm) and the BIPY^{•+} ... BIPY^{•+} supramolecular dimer (800-900 nm). By contrast, the tetracationic tetraradical states, only display NIR bands centered around 1070 nm. These observations indicate that both the mono- and bisradical states are “mixed-valence” ones. Since all four BIPY units are so closely stacked (distances < 3.6 Å), π-overlap and electronic communication exists between all four BIPY units. Consequently, the unpaired electrons of the radicals are shared by the four BIPY units to form mixed-valence states with significantly narrow bandgaps.

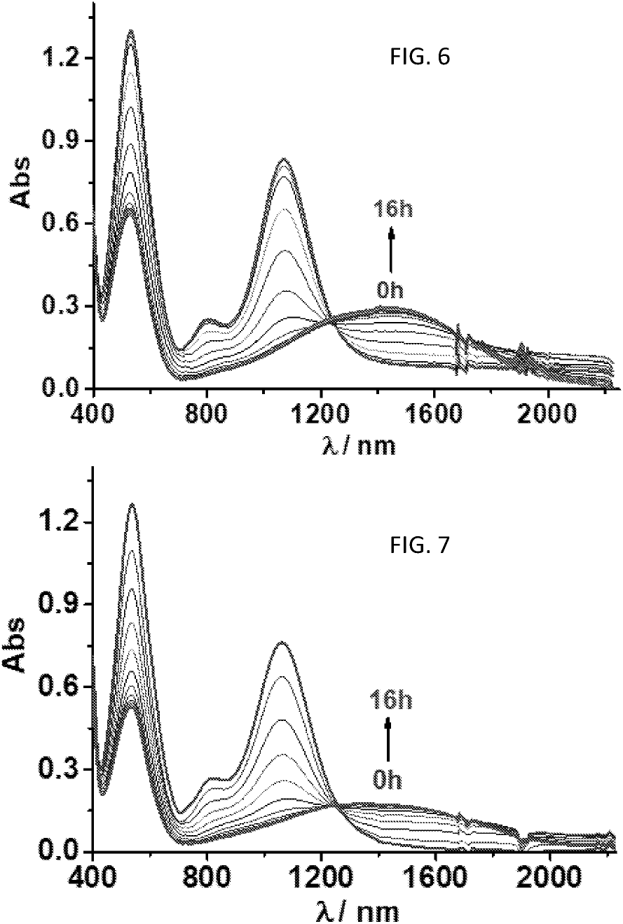

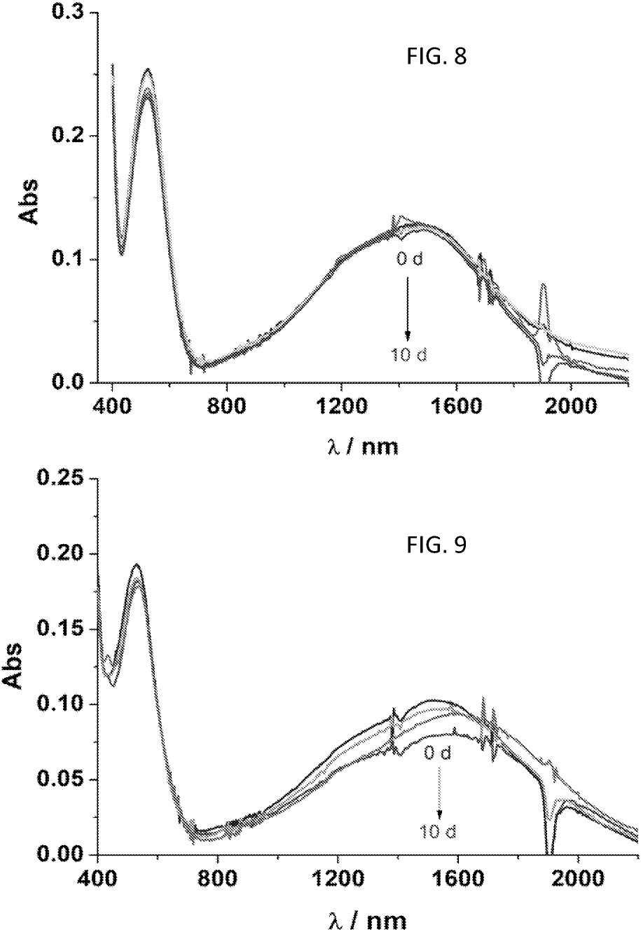

We also examined the stabilities of the tetra-, bis-, and monoradical states of the two [2]catenanes in air by time-dependent UV-Vis-NIR spectroscopy under ambient conditions. Upon exposure to air, the tetraradicals (mHe[2]C^{4(•+)} and mHo[2]C^{4(•+)}) in MeCN were observed (FIGS. 6 and 7) to decay gradually to their bisradical states (mHe[2]C^{2•6+} and mHo[2]C^{2•6+}) over several hours. The bisradicals (mHe[2]C^{2•6+} and mHo[2]C^{2•6+}), however, exhibit extraordinary stabilities under ambient conditions. MeCN solutions of mHe[2]C^{2•6+} and mHo[2]C^{2•6+} can be stored in air for more than ten days without any change (FIGS. 8 and 9) in their absorption spectra. Noteworthy is the fact that, although the monoradicals (mHe[2]C^{•7+} and mHo[2]C^{•7+}) are quite stable in their solid states, in some cases, they tend to be reduced into bisradicals in their solution states when stored in air for more than 1 weeks.

Organic NIR dyes with absorption bands longer than 1200 nm are not abundant^{3b,21}, not only because such red-shifted absorptions are difficult to achieve, but also because organic compounds with extremely narrow bandgaps suffer from stability issues. Hence, our results show that, the mono- and bisradical states of these two [2]catenanes are promising air-stable NIR-absorbing dyes with significantly red-shifted absorption peaks of ca. 1800 and 1450 nm.

The solution of two catenanes with different redox states were prepared by employing electrochemical reductions under different potentials: mHe[2]C•6PF₆ (6.3 mg) was dissolved in MeCN (30 mL) in a N₂-filled glovebox. The solution was then added into the working cell, while the auxiliary electrode chamber was filled with excess of CuPF₆(MeCN)₄ dissolved in 0.1 M TBAPF₆/MeCN solution (1 mL). The auxiliary electrode was made with a platinum wire wrapped with copper wire (diam. 0.25 mm, 99.999% trace metals basis from Sigma Aldrich). The whole apparatus was subjected to different potentials of -0.50, +0.10, and +0.42 V (vs Ag/AgCl), respectively. After retaining the each potentials for 10 min, 1 mL of each solution was drawn out from the working cell corresponding to +4, +6, and +7 states of mHe[2]C•nPF₆ respectively. Each of the three solutions was injected into a 2-mm path cuvette which sealed by Teflon caps and then was analyzed by using Vis/NIR spectroscopy. For mHe[2]C·nPF₆, the +4, +6, and +7 states were obtained using the same protocol under the potentials of -0.50, +0.10, and +0.50 V, respectively. The +6 states (bisradical hexacationic states) of mHo[2]C·nPF₆ and mHe[2]C·nPF₆ were exposed to air for several days and the Vis/NIR spectra were recorded to test their air-stability.

### EPR Spectroscopy

We also recorded (FIGS. 4C-4D) the electron paramagnetic resonance (EPR) spectra of the bisradicals (mHe[2]C^{2·6+} and mHo[2]C^{2·6+}) in MeCN. The very weak signals, which were observed, are almost negligible compared with the signal intensities of the monoradicals (FIGS. 4C-4D) recorded under similar conditions. These observations are in accordance with previously reported^{16b} results, indicating that the unpaired electrons in the bisradicals are coupled antiferromagnetically and exist as ground-state singlets. The relatively weak signals observed in the EPR spectra can be attributed to the thermally populated triplet states of the bisradicals.

### DFT Calculations

DFT calculations were performed in order to probe the electronic properties of the two catenanes. The results illustrate (FIGS. 5A-5D) that the spin densities in both mono- (mHe[2]C^{·7+} and mHo[2]C^{·7+}) and bisradical (mHe[2]C^{2·6+} and mHo[2]C^{2·6+}) states are located on their two innermost BIPY^{2+/·+} units in accordance with the experimental results. The theoretical association energies (Table S1) for the formation of catenanes in their different redox states (mHe[2]C^{(8-n)·n+} and mHo[2]C^{(8-n)·n+}, where n refers to the number of positive charges) from the corresponding cyclophanes were calculated in MeCN at the M06/6-311++G** level taking previous reported Ho[2]C^{(8-n)·n+} as the control molecule. For each of nine redox states, the theoretical association energy (ΔE) of mHe[2]C^{(8-n)·n+} and mHo[2]C^{(8-n)·n+} are always higher than the corresponding Ho[2]C^{(8-n)·n+}, indicating that the introduction of mCBPQT ring into [2]catenanes is more energetically unfavorable than it is when the ring is CBPQT because of the smaller cavity size of mCBPQT ring. Nevertheless, if we consider the binding energy difference (ΔΔE) between 7+ and 8+ state (ΔΔE = ΔE₇₊ - ΔE₈₊), or 6+ and 8+ state (ΔΔE = ΔE₆₊ - ΔE₈₊), a value which indicates the thermodynamic tendency of forming the 7+ (monoradical) or 6+ (bisradical) states from the reduction of state 8+, the two new mCBPQT-ring containing catenanes become energetically more favorable (Table S2) than that for Ho[2]C^{(8-n)·n+}. Accordingly, the mono- and bisradical forms of mCBPQT-ring-based [2]catenanes should exhibit enhanced stabilities compared with CBPQT-ring-based [2]catenane, an observation which is also in agreement with the experimental results.

Calculations were performed using density functional theory (DFT) with the M06 functional, as implemented^{S5} in Jaguar 7.6.110. Geometry optimizations were performed^{S6} using the 6-31G* basis set. Electronic energies (Table 1) were obtained^{S7} using the 6-311++G** basis set. ΔE is the difference in energy between the sum of the individual macrocycles of a particular change state and the corresponding [2]catenane of charge (8-n)·n+. Association energy differences (ΔΔE) detween different charged dtates are shown in Table 2. Solvent corrections were based on single point self-consistent Poisson-Boltzmann continuum solvation calculations for MeCN (ε = 37.5 and R₀= 2.179 Å) using^{S8} the PBF module in Jaguar.

TABLE 1

Calculated Association Energies (ΔE) of [2]Catenanes

| Charged State | ΔE (Ho[2]C) / (kcal mol⁻¹) | ΔE (mHe[2]C) / (kcal mol⁻¹) | ΔE (mHo[2]C) / (kcal mol⁻¹) |
|---|---|---|---|
| 0 | -29.0 | -25.5 | -20.6 |
| 1+ | -42.3 | -40.5 | -35.6 |
| 2+ | -50.5 | -48.7 | -44.8 |
| 3+ | -59.7 | -58.2 | -55.0 |
| 4+ | -64.1 | -60.0 | -55.7 |
| 5+ | -49.0 | -41.6 | -39.0 |
| 6+ | -26.8 | -14.6 | -12.5 |
| 7+ | -0.40 | 9.3 | 13.5 |
| 8+ | 40.2 | 55.6 | 58.9 |

TABLE 2

Calculated Association Energy Differences (ΔΔE) Between Different Charged States

| | ΔΔE (Ho[2]C) / (kcal mol⁻¹) | ΔΔE (mHe[2]C) / (kcal mol⁻¹) | ΔΔE (mHo[2]C) / (kcal mol⁻¹) |
|---|---|---|---|
| 8+ to 7+^{a} | -44.2 | -46.3 | -45.4 |
| 8+ to 6+^{b} | -67 | -70.2 | -71.4 |

^{a}ΔΔE = ΔE₇₊-ΔE₈₊; ^{b}ΔΔE = ΔE₆₊-ΔE₈₊

### REFERENCES

(1) (a) Sun, Z.; Wu, J. Open-shell polycyclic aromatic hydrocarbons. J. Mater. Chem. 2012, 22, 4151-4160; (b) Abe, M. Diradicals. Chem. Rev. 2013, 113, 7011-7088; (c) Tan, G.; Wang, X. Isolable bis(triarylamine) dications: analogues of Thiele’s, Chichibabin’s, and Muller’s hydrocarbons. Acc. Chem. Res. 2017, 50, 1997-2006.
(2) (a) Konishi, A.; Hirao, Y; Nakano, M.; Shimizu, A.; Botek, E.; Champagne, B.; Shiomi, D.; Sato, K.; Takui, T.; Matsumoto, K.; Kurata, H. Synthesis and characterization of teranthene: A singlet biradical polycyclic aromatic hydrocarbon having Kekule’ structures. J. Am. Chem. Soc. 2010, 132, 11021-110239; (b) Pan, X.; Su, Y; Chen, X.; Zhao, Y; Li, Y; Zuo, J.; Wang, X. Stable tetraaryldiphosphine radical cation and dication. J Am. Chem. Soc. 2013, 135, 5561-5564; (c) Schmidt, D.; Bialas, D.; Würthner, F. Ambient stable zwitterionic perylene bisimide-centered radical. Angew. Chem. Int. Ed. 2015, 54, 3611-3614; (d) Kumar, Y; Kumar, S.; Mandal, K.; Mukhopadhyay, P. Isolation of tetracyano-naphthalenediimide and its stable planar radical anion. Angew. Chem. Int. Ed. 2018, 57, 16570-16570; (e) Zeng, W.; Gopalakrishna, T. Y, ; Phan, H.; Tanaka, T.; Herng, T. S.; Ding, J.; Osuka, A.; Wu, J. Superoctazethrene: an open-shell graphene-like molecule possessing large diradical character but still with reasonable stability. J. Am. Chem. Soc. 2018, 140, 43, 14054-14058; (f) Rana, A.; Hong, Y; Gopalakrishna, T. Y; Phan, H.; Herng, T. S.; Yadav, P.; Ding, J.; Kim, D. Wu, J. Stable expanded porphycene-based diradicaloid and tetraradicaloid. Angew. Chem. Int. Ed. 2018, 57, 12534-12537; (g) Jiao, T.; Cai, K.; Nelson, J.N.; Jiao, Y; Qiu, Y; Wu, G.; Zhou, J.; Cheng, C.; Shen, D.; Feng, Y Liu, Z.; Wasielewski, M. R.; Stoddart, J. F.; Li, H. Stabilizing the naphthalenediimide radical within a tetracationic cyclophane. J. Am. Chem. Soc. 2019, 141, 16915-16922.
(3) (a) Peng, Q.; Obolda, A.; Zhang, M.; Li, F. Organic light-emitting diodes using a neutral π radical as emitter: The emission from a doublet. Angew. Chem. Int. Ed. 2015, 54, 7091-7095; (b) Ni, Y; Wu. J. Diradical approach toward organic near infrared dyes. Tetrahedron Lett. 2016, 57, 5426-5434; (c) Ai, X.; Evans, E. W.; Dong, S.; Gillett, A. J.; Guo, H.; Chen, Y.; Hele, T. J.; Friend, R.H.; Li, F. Efficient radical-based light-emitting diodes with doublet emission. Nature, 2018, 563, 536.
(4) (a) Joo, Y; Agarkar, V.; Sung, S. H.; Savoie, B. M.; Boudouris, B. W. A nonconjugated radical polymer glass with high electrical conductivity. Science, 2018, 359, 1391-1395; (b) Wang, P.; Lin, S.; Lin, Z.; Peeks, M. D.; Van Voorhis, T.; Swager, T. M. A semiconducting conjugated radical polymer: Ambipolar redox activity and Faraday Effect. 2018, 140, 34, 10881-10889; (c) London, A. E.; Chen, H.; Sabuj, M. A.; Tropp, J.; Saghayezhian, M.; Eedugurala, N.; Zhang, B. A.; Liu, Y; Gu, X.; Wong, B. M.; Rai, N. A high-spin ground-state donor-acceptor conjugated polymer. Sci. Ad. 2019, 5, No. eaav2336.
(5) (a) Mahmood, J.; Park, J.; Shin, D.; Choi, H. J.; Seo, J. M.; Yoo, J. W.; Baek, J. B.; Organic ferromagnetism: trapping spins in the glassy state of an organic network structure. Chem, 2018, 4, 2357-2369; (b) Sugawara, T.; Komatsu, H.; Suzuki, K.; Interplay between magnetism and conductivity derived from spin-polarized donor radicals. Chem. Soc. Rev. 2011, 40, 3105-3118; (b) Phan, H.; Herng, T. S.; Wang, D.; Li, X.; Zeng, W.; Ding, J.; Loh, K. P.; Wee, A. T. S.; Wu, J. Room-temperature magnets based on 1,3,5-triazine-linked porous organic radical frameworks. Chem, 2019, 5, 1223-1234.
(6) (a) Chen, X.; Wang, X.; Zhou, Z.; Li, Y; Sui, Y; Ma, J.; Wang, X.; Power, P. P. Reversible σ-dimerizations of persistent organic radical cations. Angew. Chem. Int. Ed. 2013, 52, 589-592; (b) Zhao, D.; Fu, X. Cyclo-oligomerization of 6,12-diethynyl indeno[1,2-b]fluorenes via diradical intermediates. Org. Lett. 2015, 17, 5694-5697; (c) Okino, K.; Hira, S.; Inoue, Y; Sakamaki, D.; Seki, S. The divergent dimerization behavior of N-Substituted dicyanomethyl radicals: dynamically stabilized versus stable radicals. Angew. Chem. Int. Ed. 2017, 56, 16597-16601.
(7) (a) Zeng, Z.; Sung, Y M.; Bao, N.; Tan, D.; Lee, R.; Zafra, J. L.; Lee, B. S.; Ishida, M.; Ding, J.; López Navarrete, J. T.; Li, Y Stable tetrabenzo-Chichibabin’s hydrocarbons: tunable ground state and unusual transition between their closed-shell and open-shell resonance forms. J. Am. Chem. Soc. 2012, 134, 14513-14525. (b) Tian, Y; Uchida, K.; Kurata, H.; Hirao, Y; Nishiuchi, T.; Kubo, T. Design and synthesis of new stable fluorenyl-based radicals. J. Am. Chem. Soc. 2014, 136, 12784-12793.
(8) Kumar, S.; Ajayakumar, M.R.; Hundal, G.; Mukhopadhyay, P. Extraordinary stability of naphthalenediimide radical ion and its ultra-electron-deficient precursor: Strategic role of the phosphonium group. J Am. Chem. Soc. 2014, 136, 12004-12010.
(9) (a) Zeng, Z.; Shi, X.; Chi, C.; Navarrete, J. T. L.; Casado, J.; Wu, J. Pro-aromatic and anti-aromatic π-conjugated molecules: An irresistible wish to be diradicals. Chem. Soc. Rev. 2015, 44, 6578-6596; (b) Liu, C.; Ni, Y; Lu, X.; Li, G.; Wu, J. Global aromaticity in macrocyclic polyradicaloids: Hückel’s Rule or Baird’s Rule? Acc. Chem. Res. 2019, 52, 2309-2321.
(10) (a) Michaelis, L.; Hill, E.S. The viologen indicators. J. Gen. Physiol. 1933. 16, 859-873; (b) Hünig, S. Stable radical ions. Pure Appl. Chem. 1967, 15, 109-122; (c) Kosower, E. M.; Hajdu, J. Pyridinyl diradical pimer. Magnesium iodide complexes. J. Am. Chem. Soc. 1971, 93, 2534-2535; (d) Bird, C. L.; Kuhn, A. T. Electrochemistry of the viologens. Chem. Soc. Rev. 1981, 10, 49-82; (e) Bockman, T. M.; Kochi, J. K. Isolation and oxidation-reduction of methylviologen cation radicals. Novel disproportionation in charge-transfer salts by X-ray crystallography. J. Org. Chem. 1990, 55, 4127-4135.
(11) (a) Jeon, W.S.; Kim, H. J.; Lee, C.; Kim, K. Control of the stoichiometry in host-guest complexation by redox chemistry of guests: Inclusion of methylviologen in cucurbituril. Chem. Commun. 2002, 1828-1829; (b) Iordache, A.; Oltean, M.; Milet, A.; Thomas, F.; Baptiste, B.; Saint-Aman, E.; Bucher, C. Redox control of rotary motions in ferrocene-based elemental ball bearings. J. Am. Chem. Soc. 2012, 134, 2653-2671; (c) Buck, A. T.; Paletta, J. T.; Khindurangala, S. A.; Beck, C. L.; Winter, A. H. A noncovalently reversible paramagnetic switch in water. J. Am. Chem. Soc. 2013, 135, 10594-10597; (d) Zhang, L.; Zhou, T. Y; Tian, J.; Wang, H.; Zhang, D. W.; Zhao, X.; Liu, Y; Li, Z. -T. Poly. Chem. 2014, 5, 4715-4721.
(12) (a) Moon, K.; Grindstaff, J.; Sobransingh, D.; Kaifer, A. E. Cucurbituril-mediated redox-controlled self-assembly of viologen-containing dendrimers. Angew. Chem. Int. Ed. 2004, 5496-5499; (b) Zhang, D.W.; Tian, J.; Chen, L.; Zhang, L.; Li, Z.-T. Dimerization of conjugated radical cations: An emerging non-covalent interaction for self-assembly. Chem. Asian. J. 2015, 10, 56-68; (c) Cai, K.; Lipke, M.C.; Liu, Z.; Nelson, J.; Cheng, T.; Shi, Y; Cheng, C.; Shen, D.; Han, J. -M.; Vemuri, S.; Feng, Y; Stern, C. L.; Goddard III, W. A.; Wasielewski, W. R.; Stoddart, J. F. Molecular Russian dolls, Nat. Commun. 2018, 9, 5275.
(13) Li, H.; Fahrenbach, A. C.; Dey, S. K.; Basu, S.; Trabolsi, A.; Zhu, Z.; Botros, Y Y; Stoddart, J. F. Mechanical bond formation by radical templation. Angew. Chem. Int. Ed. 2010, 49, 8260-8265.
(14) Berville, M.; Richard, J.; Stolar, M.; Choua, S.; Le Breton, N.; Gourlaouen, C.; Boudon, C.; Ruhlmann, L.; Baumgartner, T.; Wytko, J. A.; Weiss, J. A highly stable organic radical cation. Org. Lett. 2018, 20, 8004-8008.
(15) Trabolsi, A.; Khashab, N.; Fahrenbach, A. C.; Friedman, D. C.; Colvin, M. T.; Cotí, K. K.; Benítez, D.; Tkatchouk, E.; Olsen, J. C.; Belowich, M. E.; Carmielli, R. H.; Khatib, A.; Goddard III, W. A.; Wasielewski M. R.; Stoddart, J. F. Radically enhanced molecular recognition. Nat. Chem. 2010, 2, 42-49.
(16) (a) Li, H.; Zhu, Z.; Fahrenbach, A. C.; Savoie, B. M.; Ke, C.; Barnes, J. C.; Lei, J.; Zhao, Y L.; Lilley, L. M.; Marks, T. J.; Ratner, M. A. Mechanical bond-induced radical stabilization. J. Am. Chem. Soc. 2012, 135, 456-467; (b) Barnes, J. C.; Fahrenbach, A. C.; Cao, D.; Dyar, S. M.; Frasconi, M.; Giesener, M. A.; Benítez, D.; Tkatchouk, E.; Chernyashevskyy, O.; Shin, W. H.; Li, H.; Sampath, S.; Stern, C. L.; Sarjeant, A. A.; Hartlieb, K. J.; Liu, Z.; Carmieli, R.; Botros, Y. Y; Choi, J. W.; Slawin, A. M. Z.; Ketterson, J. B.; Wasielewski, M. R.; Goddard III, W. A.; Stoddart, J. F. A radically configurable six-state compound. Science 2013, 339, 429-433; (c) Sun, J.; Liu, Z.; Liu, W. G.; Wu, Y; Wang, Y; Barnes, J. C.; Hermann, K. R.; Goddard III, W. A.; Wasielewski, M. R.; Stoddart, J. F. Mechanical-bond-protected, air-stable radicals. J. Am. Chem. Soc. 2017, 139, 12704-12709.
(17) Cai, K.; Shi, Y; Vemuri, S.; Cao, C.; Cui, B.; Shen, D.; Wu, H.; Zhang, L.; Qiu, Y; Chen, H.; Jiao, Y; Stern, C. L.; Alsubaie, F. M.; Xiao, H.; Li, J.; Stoddart, J. F. Tuning radical interactions in trisradical tricationic complexes by varying host-cavity sizes. Chem. Sci. 2019, 11, 107-112.
(18) NA
(19) NA
(20) Wang, Z.; Qian, G. Near-infrared organic compounds and emerging applications. Chem. Asian J. 2010, 5, 1006-1029.
(21) Sun, Z.; Ye, Q.; Chi, C.; Wu, J. Low band gap polycyclic hydrocarbons: from closed-shell near infrared dyes and semiconductors to open-shell radicals. Chem. Soc. Rev. 2012, 41, 7857-7889.
(22) (a) Lü, B.; Chen, Y; Li, P.; Wang, B.; Müllen, K.; Yin, M. Stable radical anions generated from a porous perylenediimide metal-organic framework for boosting near-infrared photothermal conversion. Nat. Commun. 10, 767; (b) Tang, B.; Li, W. L.; Chang, Y; Yuan, B.; Wu, Y; Zhang, M. T.; Xu, J. F.; Li, J.; Zhang, X. A supramolecular radical dimer: High-efficiency NIR-II photothermal conversion and therapy. Angew. Chem. Int. Ed. 2019, 58, 15526-15531.
(23) (a) Yao, C. J.; Zhong, Y W.; Nie, H. J.; Abruna, H. D.; Yao, J. Near-IR electrochromism in electropolymerized films of a biscyclometalated ruthenium complex bridged by 1, 2, 4, 5-tetra (2-pyridyl) benzene. J. Am. Chem. Soc. 2011, 133, 20720-20723; (b) Cai, K.; Yan, Q.; Zhao, D. Large hydroazaacene diimides: synthesis, tautomerism, halochromism, and redox-switchable NIR optics, Chem. Sci. 2012, 3, 3175-3182; (c) Keshri, S. K.; Asthana, D.; Chorol, S.; Kumar, Y; Mukhopadhyay, P. Appending diverse π-extended acceptors with tetrathiafulvalene/dithiafulvalene donors: multistate redox properties, radical ion generation, and mid-IR-absorbing mixed-valence states. Chem. Eur. J. 2018, 24, 1821-1832.
(24) Cui, B. B.; Tang, J. H.; Yao, J.; Zhong, Y W. A molecular platform for multistate near-infrared electrochromism and flip-flop, flip-flap-flop, and ternary memory. Angew. Chem. Int. Ed. 2015, 54, 9192-9197
(S1) Cai, K.; Shi, Y.; Vemuri, S.; Cao, C.; Cui, B.; Shen, D.; Wu, H.; Zhang, L.; Qiu, Y.; Chen, H.; Jiao, Y.; Stern, C. L.; Alsubaie, F. M.; Xiao, H.; Li, J.; Stoddart, J. F. Tuning radical interactions in trisradical tricationic complexes by varying host-cavity sizes. Chem. Sci. 2020, 11, 107-112
(S2) Dolomanov, O. V.; Bourhis, L. J.; Gildea, R. J.; Howard, J. A. K.; Puschmann, H. OLEX2: a complete structure solution, refinement and analysis program. J. Appl. Cryst. 2009, 42, 339-341.
(S3) Sheldrick, G. M. Crystal structure refinement with SHELXL. Acta Cryst. 2015, A71, 3-8.
(S4) Sheldrick, G. M. A short history of SHELX. Acta Cryst. 2008, A64, 112-122.
(S5) Jaguar 7.6, Schrödinger, LLC, New York, NY (2006).
(S6) Krishnan, R.; Binkley, J. S.; Seeger, R.; Pople, J. A. Self - consistent molecular orbital methods. XX. A basis set for correlated wave functions. J. Chem. Phys. 1980, 72, 650-654.
(S7) Frisch, M. J.; Pople, J. A.; Binkley, J. S. Self - consistent molecular orbital methods 25. Supplementary functions for Gaussian basis sets. J. Chem. Phys. 1984, 80, 3265-3269.
(S8) Tannor, D. J.; Marten, B.; Murphy, R.; Friesner, R. A.; Sitkoff, D.; Nicholls, A.; Honig, B.; Ringnalda, M.; Goddard, W. A., III Accurate first principles calculation of molecular charge distributions and solvation energies from ab initio quantum mechanics and continuum dielectric theory. J. Am. Chem. Soc. 1994, 116, 11875-11882.

1. A composition comprising a [2]catenane, the [2]catenane comprising a first macrocyclic ring mechanically interlocked with a second macrocyclic ring, or a salt thereof, wherein each of the first macrocyclic ring and the second macrocyclic ring comprise an alternating cyclic arrangement of a first unsubstituted or substituted 4,4′-bipyridinium (BIPY) subunit, a first unsubstituted or substituted phenylene subunit, a second unsubstituted or substituted BIPY subunit, and a second unsubstituted or substituted phenylene subunit forming a macrocycle, wherein the first macrocyclic ring comprises a meta-phenylene subunit.

2. The composition of claim 1, wherein the composition comprises an air-stable bisradical/hexacation or an air-stable monoradical/heptacation.

3. The composition of claim 2, wherein the composition comprises the air-stable bisradical/hexacation.

4. The composition of claim 1, wherein the first ring or both the first ring and the second ring are mCBPQT.

5. The composition of claim 4, wherein both the first ring and the second ring are mCBPQT.

6. The composition of claim 4, wherein the first ring is mCBPQT.

7. The composition of claim 6, wherein the second ring is CBPQT.

8. The composition of claim 1, wherein the composition has an E_{red1} greater than +0.50 V versus Ag/AgCl.

9. The composition of claim 1, wherein the composition has an E_{red2} greater than +0.25 V versus Ag/AgCl.

10. The composition of claim 1, wherein the composition has a near infrared absorption band longer than 1200 nm.

11. (canceled)

12. A crystalline composition comprising the composition of claim 1 having a molecular packing arranging defined by a triclinic, space group P1‾(no. 2) or a orthorhombic, space group Pna2₁ (no. 33).

13. The crystalline composition of claim 12, wherein the composition has a molecular packing arranging defined by the triclinic, space group P1‾(no. 2) and lattice parameters of a = 14.1 ± 0.1 Å, b = 15.3 ± 0.1 Å, c = 23.3 ± 0.1 Å, α = 84.6 ± 0.1°, β = 82.2 ± 0.1°, and γ = 66.5 ± 0.1°.

14. The crystalline composition of claim 12, wherein the composition has a molecular packing arranging defined by the orthorhombic, space group Pna2₁ (no. 33) and lattice parameters of a = 27.2 ± 0.1 Å, b = 20.4 ± 0.1 Å, and c = 16.7 ± 0.1 Å .

15. The crystalline composition of claim 12, wherein the composition comprises six counter anions for every [2]catenane.

16. (canceled)

17. (canceled)

18. A method for preparing a [2]catenane, the method comprising:
(a) contacting a cationic ring with a cationic guest molecule in the presence of a reducing agent, thereby reducing the cationic ring and the cationic guest molecule and forming a radical cationic inclusion complex; and
(b) reacting the guest molecule of the radical cationic inclusion complex with a ring-closing reagent to prepare the [2]catenane or reaching the termini of the guest molecule of the radical cationic inclusion complex with each other to prepare the [2]catenane.

19. (canceled)

20. The method of claim 18, wherein the cationic ring is mCBPQT⁴⁺.

21. The method of claim 18, wherein the cationic guest molecule is 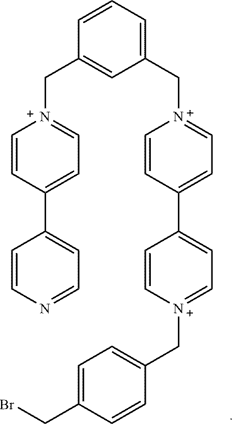
.

22. The method of claim 18, wherein the cationic guest molecule is 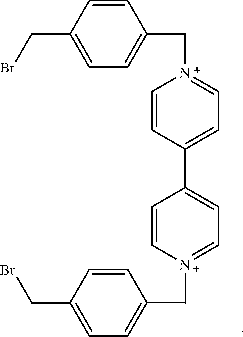
.

23. The method of claim 22, wherein the ring-closing reagent is 4,4′-bipyridine.

24. The method of claim 18 further comprising reducing the [2]catenane with a second reducing agent to prepare a reduced [2]catenane.
